(19) United States
(12) Patent Application Publication
(10) Pub. No.: US 2017/0130170 A1
(43) Pub. Date: May 11, 2017

(54) Dishwashing Composition Containing Cellulytic Enzymes and Use Thereof

(71) Applicant: Novozymes A/S, Bagsvaerd (DK)

(72) Inventor: Jan Peter Skagerlind, Hellsingborg (SE)

(73) Assignee: Novozymes A/S, Bagsvaerd (DK)

(21) Appl. No.: 15/127,451

(22) PCT Filed: Mar. 25, 2015
(86) PCT No.: PCT/EP2015/056453

§ 371 Date: Sep. 20, 2016

(30) Foreign Application Priority Data
Mar. 25, 2014 (EP) 14161558.3
Sep. 17, 2014 (EP) 14185198.0

(51) Int. Cl. C11D 3/386; C12N 9/42; C12N 9/24; C11D 11/00

(52) U.S. Cl. CPC C11D 3/38636; C11D 3/38645; C11D 11/0023; C12N 9/2437; C12N 9/2445; C12N 9/2402; C12Y 302/01091; C12Y 302/01021

(57) ABSTRACT

Dishwashing composition comprising an enzyme preparation, which enzyme preparation comprises enzymes capable of degrading cellulosic material. The enzyme preparation may comprise: a) an Aspergillus fumigatus cellobiohydrolase I; b) an Aspergillus fumigatus cellobiohydrolase II; c) an Aspergillus fumigatus beta-glucosidase or variant thereof; and d) a Penicillium sp. GH61 polypeptide having cellulolytic enhancing activity; or homologs thereof. The dishwashing composition can be used in a method for automatic dishwashing for example in combination with an acidic material or it can be used for cleaning the interior of the dishwashing machine. The addition of a non-pathogenic microorganism, e.g. Bacillus subtilis, to the dishwashing detergent is also suggested.

### FIELD OF THE INVENTION

The present invention concerns dishwashing composition comprising an enzyme capable of degrading cellulosic material or a cleaning composition comprising such enzyme. The invention further concerns a dishwashing method, the use of enzymes capable of degrading cellulosic material for dishwashing or cleaning, and a rinsing aid comprising an enzyme capable of degrading cellulosic material.

### REFERENCE TO A SEQUENCE LISTING

This application contains a Sequence Listing in computer readable form. The computer readable form is incorporated herein by reference.

### BACKGROUND OF THE INVENTION

Use of enzymes in dishwashing detergents is well known in the field of both automatic dishwashing (ADW) formulas, and liquid hand dishwashing formulas (LDLs). Typically proteases and amylases are used in commercial dishwashing detergents. These enzymes are useful for degrading protein and starch/amylose, respectively.

The degradation of cellulosic material in dishwashing machines is often a challenge. The consumer very often does not rinse the tableware before putting it into the dishwasher. This poses a problem, when the consumer eats foods like salad, spinach or other leaves. The remains of the leaves on the dish is not removed before the dish is put in the dishwasher. As the commercial dishwashing detergents usually contain proteases and amylases, the cellulosic material like salad is not degraded during the washing process. This results in salad sticking to the dishes which is then difficult to remove. Further the remains of the leaves will be left in the drain of the dishwasher after use. This results in a poorer washing result and the drain therefore needs to be cleaned manually from time to time.

### SUMMARY OF THE INVENTION

The present invention concerns a dishwashing composition comprising one or more enzymes capable of degrading cellulosic material, a washing method comprising exposing the dish ware to wash liquor comprising one or more enzymes capable of degrading cellulosic material, the use of the enzyme capable of degrading cellulosic material in a dishwashing process, a cleaning method for cleaning the interior of an automated dish washing machine, a rinsing aid and use of the rinsing aid.

### BRIEF DESCRIPTION OF THE FIGURES

FIG. 1 shows the temperature during the ADW wash.

### DEFINITIONS

The term automatic dishwashing composition refers to compositions intended for cleaning dishware such as plates, cups, glasses, bowls, cutlery such as spoons, knives, forks, serving utensils, ceramics, plastics, metals, china, glass and acrylics in a dishwashing machine. The terms encompass any materials/compounds selected for domestic or industrial washing applications and the form of the product can be liquid, powder or granulate. In addition to lipase, the automatic dishwashing composition contains detergent components such as polymers, bleaching systems, bleach activators, bleach catalysts, silicates, dyestuff and metal care agents.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20 (polyoxyethylene sorbitan monolaurate).

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. For purposes of the present invention, one unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TVVEEN® 20.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teen et al., 1998, Trichoderma reesei cellobiohydrolases: why so efficient on crystalline cellulose?, Biochem. Soc. Trans. 26: 173-178). Cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Tomme et al. method can be used to determine cellobiohydrolase activity.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-50 mg of cellulolytic enzyme protein/g of cellulose in PCS (or other pretreated cellulosic material) for 3-7 days at a suitable temperature, e.g., 50° C., 55° C., or 60° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in vegetable food products, such as salad, tomatoes, spinach, cabbage, grain or the like.

Detergent components: The term “detergent components” is defined herein to mean the types of chemicals which can be used in detergent compositions for automatic dishwashing. Examples of detergent components are polymers, bleaching systems, bleach activators, bleach catalysts, silicates, dyestuff and metal care agents.

Dishware: The term dish ware is intended to mean any form of kitchen utensil, dinner set or tableware such as but not limited to pans, plates, cops, knives, forks, spoons, porcelain etc.

Dish washing composition: The term “dish washing composition” refers to compositions comprising detergent components, which composition is intended for cleaning dishes, table ware, pots, pans, cutlery and all forms of compositions for cleaning hard surfaces areas in kitchens. The present invention is not restricted to any particular type of dish wash composition or any particular detergent.

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Fragment: The term “fragment” means a polypeptide having one or more (e.g., several) amino acids absent from the amino and/or carboxyl terminus of a mature polypeptide main; wherein the fragment has enzyme activity. In one aspect, a fragment contains at least 85%, e.g., at least 90% or at least 95% of the amino acid residues of the mature polypeptide of an enzyme.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Improved wash performance: The term “improved wash performance” is defined herein as an automatic dishwashing detergent composition displaying an increased wash performance relative to the wash performance of a similar automatic dishwashing detergent composition without the inventive enzyme preparation, e.g. by increased soil removal.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “parent subtilase” describes a subtilase defined according to Siezen et al. (1991 and 1997). For further details see description of “Subtilases” above. A parent subtilase may also be a subtilase isolated from a natural source, wherein subsequent modifications have been made while retaining the characteristic of a subtilase. Furthermore, a parent subtilase may be a subtilase which has been prepared by the DNA shuffling technique, such as described by J. E. Ness et al., Nature Biotechnology, 17, 893-896 (1999).

Alternatively the term “parent subtilase” may be termed “wild type subtilase”.

Polypeptide having cellulolytic enhancing activity: The term “polypeptide having cellulolytic enhancing activity” means a GH61 polypeptide that catalyzes the enhancement of the hydrolysis of a cellulosic material by enzyme having cellulolytic activity. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions: 1-50 mg of total protein/g of cellulose in PCS, wherein total protein is comprised of 50-99.5% w/w cellulolytic enzyme protein and 0.5-50% w/w protein of a GH61 polypeptide having cellulolytic enhancing activity for 1-7 days at a suitable temperature, e.g., 50° C., 55° C., or 60° C., and pH, e.g., 5.0 or 5.5, compared to a control hydrolysis with equal total protein loading without cellulolytic enhancing activity (1-50 mg of cellulolytic protein/g of cellulose in PCS). In a preferred aspect, a mixture of CELLUCLAST® 1.5 L (Novozymes A/S, Bagsværd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptides having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Pretreated corn stover: The term “PCS” or “Pretreated Corn Stover” means a cellulosic material derived from corn stover by treatment with heat and dilute sulfuric acid, alkaline pretreatment, or neutral pretreatment.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having enzyme activity. In one aspect, a subsequence contains at least 85%, e.g., at least 90% or at least 95% of the nucleotides of the mature polypeptide coding sequence of an enzyme.

Variant: The term “variant” means a polypeptide having enzyme activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Wash cycle: The term “wash cycle” is defined herein as a washing operation wherein dishware are exposed to the wash liquor for a period of time by circulating the wash liquor and spraying the wash liquor onto the dishware in order to clean the dishware and finally the superfluous wash liquor is removed. A wash cycle may be repeated one, two, three, four, five or even six times at the same or at different temperatures. Hereafter the dishware is generally rinsed and dried. One of the wash cycles can be a soaking step, where the dishware is left soaking in the wash liquor for a period.

Wash liquor: The term “wash liquor” is intended to mean the solution or mixture of water and detergents optionally including enzymes used for dishwashing.

Wash performance: The term “wash performance” is defined herein as the ability of an automatic dishwashing detergent composition to remove soil present on dishware to be cleaned during washing. The wash performance may be measured by inspecting the washed dishware, light reflectance (460 nm) or by measuring weight, how much of the soil has been removed. This can be done by measuring the difference in weight on plates, tiles or similar.

Wash time: The term “wash time” is defined herein as the time it takes for the entire washing process; i.e. the time for the wash cycle(s) and rinse cycle(s) together.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, Recent progress in the assays of xylanolytic enzymes, 2006, Journal of the Science of Food and Agriculture 86(11): 1636-1647; Spanikova and Biely, 2006, Glucuronoyl esterase—Novel carbohydrate esterase produced by Schizophyllum commune, FEBS Letters 580(19): 4597-4601; Herrmann, Vrsanska, Jurickova, Hirsch, Biely, and Kubicek, 1997, The beta-D-xylosidase of Trichoderma reesei is a multifunctional beta-D-xylan xylohydrolase, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 (4-(1,1,3,3-tetramethylbutyl)phenyl-polyethylene glycol) and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

For purposes of the present invention, xylan degrading activity is determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, A new reaction for colorimetric determination of carbohydrates, Anal. Biochem 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

### DETAILED DESCRIPTION OF THE INVENTION

The present invention concerns a dishwashing composition for use in an automatic dishwashing process. The composition comprises one or more enzymes capable of degrading cellulosic material. The inventor has found that the degradation of cellulose during a dish washing process is an advantage for the degradation of cellulose containing food but also for the overall result of the washing process.

The enzymes capable of degrading cellulosic material can be selected from the group consisting of Aspergillus fumigatus GH10 xylanases, Aspergillus fumigatus beta-xylosidases, Aspergillus fumigatus cellobiohydrolase I, Aspergillus fumigatus cellobiohydrolase II, Aspergillus fumigatus beta-glucosidase variants and Penicillium sp. (emersonii) GH61 polypeptide.

The inventor has found that these enzymes are superior in degrading cellulosic material during a dishwashing process.

When dishes with remains of cellulosic material such as spinach, salad, fruit, grains or vegetables is washed in an automatic dishwashing machine, the cellulosic material is not degraded but will remain on the dishes after wash or be flushed into the drain of the dishwasher. The inventor has found that enzymes capable of degrading cellulosic material degrades the cellulosic material present on the dishes and the cellulosic material left in the drain of the dishwasher will be minimized. The dishes are therefore cleaner after being washed. Also, cellulosic material remained in drain from an earlier wash will not be redeposited on the dishes. In addition, the consumer needs not to clean manually the drain of the dishwashing machine as often as usual as no cellulosic material blocks the drain.

The inventor has found that the dishwashing composition of the invention can comprise a builder and an enzyme preparation, which enzyme preparation comprises;

(i) a cellobiohydrolase I;

(ii) a cellobiohydrolase II;

(iii) a beta-glucosidase or variant thereof; and

(iv) a GH61 polypeptide having cellulolytic enhancing activity; or homologs thereof.

The dishwashing composition according to the invention can comprise a builder and an enzyme preparation comprising one or more enzymes capable of degrading cellulosic material, which enzyme preparation comprises:
a) an Aspergillus fumigatus cellobiohydrolase I;
b) an Aspergillus fumigatus cellobiohydrolase II;
c) an Aspergillus fumigatus beta-glucosidase or variant thereof; and
d) a Penicillium sp. GH61 polypeptide having cellulolytic enhancing activity; or homologs thereof.

The Aspergillus fumigatus cellobiohydrolase I or homolog thereof of the enzyme preparation is selected from the group consisting of:
(i) a cellobiohydrolase I comprising or consisting of the mature polypeptide of SEQ ID NO: 2;
(ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
(iii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
(iv) a cellobiohydrolase I encoded by a polynucleotide that hybridizes under at least high stringency conditions, very high stringency conditions, with the mature polypeptide coding sequence of SEQ ID NO: 1 or the full-length complement thereof.

The Aspergillus fumigatus cellobiohydrolase II or homolog thereof is selected from the group consisting of:
(i) a cellobiohydrolase II comprising or consisting of the mature polypeptide of SEQ ID NO: 4;
(ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
(iii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
(iv) a cellobiohydrolase II encoded by a polynucleotide that hybridizes under at least high stringency conditions, very high stringency conditions, with the mature polypeptide coding sequence of SEQ ID NO: 3 or the full-length complement thereof.

The Aspergillus fumigatus beta-glucosidase or homolog thereof is selected from the group consisting of:
(i) a beta-glucosidase comprising or consisting of the mature polypeptide of SEQ ID NO: 6;
(ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
(iii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
(iv) a beta-glucosidase encoded by a polynucleotide that hybridizes under at least high stringency conditions, very high stringency conditions, with the mature polypeptide coding sequence of SEQ ID NO: 5 or the full-length complement thereof; and
(v) a beta-glucosidase variant comprising a substitution at one or more positions corresponding to positions 100, 283, 456, and 512 of the mature polypeptide of SEQ ID NO: 6, wherein the variant has beta-glucosidase activity; and

The Penicillium sp. GH61 polypeptide having cellulolytic enhancing activity or homolog thereof is selected from the group consisting of:
(i) a GH61 polypeptide having cellulolytic enhancing activity comprising or consisting of the mature polypeptide of SEQ ID NO: 6;
(ii) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
(iii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
(iv) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.

The beta-glucosidase variant of the enzyme preparation comprises one or more (several) substitutions selected from the group consisting of G142S, Q183R, H266Q, and D703G.

The enzyme preparation can further comprise one or more enzymes selected from the group consisting of:

an Aspergillus fumigatus xylanase or homolog thereof,

an Aspergillus fumigatus beta-xylosidase or homolog thereof; or

a combination of (i) and (ii).

The Aspergillus fumigatus xylanase or homolog thereof is selected from the group consisting of:

an Aspergillus fumigatus xylanase comprising or consisting of the mature polypeptide of SEQ ID NO: 10, SEQ ID NO: 12, or SEQ ID NO: 14;

a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;

a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and

a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof.

The Aspergillus fumigatus beta-xylosidase or homolog thereof is selected from the group consisting of:

beta-xylosidase comprising or consisting of the mature polypeptide of SEQ ID NO: 16;

a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;

a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and

a beta-xylosidase encoded by a polynucleotide that hybridizes under at least high stringency conditions, very high stringency conditions, with the mature polypeptide coding sequence of SEQ ID NO: 15 or the full-length complement thereof.

The enzymes capable of degrading cellulosic material can be present in an dishwashing composition comprising at least one more enzyme. The additional enzyme can be selected from the group consisting of: protease, lipase, cutinase, amylase, carbohydrase, cellulase, pectate lyase, pectinase, mannanase, arabinase, galactanase, and/or xylanase. In a preferred embodiment of the invention the additional enzymes are amylase and/or protease.

The amylase can be an alpha-amylase or a glucoamylase of bacterial or fungal origin. The amylase can be an alpha-amylase obtained from Bacillus, such as Bacillus licheniformis.

In one embodiment of the invention, the amylase is an alpha-amylase having SEQ ID NO: 17 or a variant thereof having at least 80%, at least 85% or at least 90% sequence identity to SEQ ID NO: 17 and having a substitution, a deletion or an insertion of one amino acids downstream for the amino acid corresponding to the positions in the amylase having SEQ ID NO: 17: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484; Particular preferred amylases include such a variant having a deletion of D183 and G184 and having the substitutions R118K, N195F, R320K and R458K and a variant additionally having substitutions in one or more positions selected from the group: M9, G149, G182, G186, M202, T257, Y295, N299, M323, A339 and E345, most preferred a variant additionally having substitutions in all these positions; or a variant alpha-amylase derived from a parent α-amylase derived from B. licheniformis comprising the mutation: A1*+N2*+L3V+M15T+R23K+S29A+A30E+Y31H+A33S+E34D+H35I+M197T.

In one embodiment of the invention, the amylase can be Stainzyme® sold by Novozymes A/S.

In one embodiment of the invention, the additional enzyme can be a protease.

In one embodiment, the protease is chemically modified or protein engineered. The protease can be a serine protease or a metalloprotease, preferably an alkaline microbial protease or a trypsin-like protease. The protease may be selected from the group consisting of Bacillus, e.g., subtilisin Novo, subtilisin Carlsberg, subtilisin 309, subtilisin 147, subtilisin 168, trypsin of bovine origin, trypsin of porcine origin and Fusarium protease.

In one embodiment, the protease has at least 90%, such as at least 95%, sequence identity to SEQ ID NO: 21. The protease has at least 90% identity to the amino acid sequence of SEQ ID NO: 21 or a variant thereof with substitutions in one or more of the following positions: 27, 36, 57, 76, 87, 97, 101, 104, 120, 123, 167, 170, 194, 206, 218, 222, 224, 235, and 274, preferably the variant is an alkaline protease having at least 90% identity to the amino acid sequence of SEQ ID NO: 21 with the following substitution: M222S or substitutions N76D+G195E. The protease is a subtilisin variant, wherein the variant comprises the substitutions 9R, 15T, 68A, 245R and 218 {D,G,V} in a parent subtilisin, and wherein the positions corresponds to the positions of the mature polypeptide of SEQ ID NO: 22 [BPN′]. In one embodiment the substitution at position 218 is with D. The protease may further comprise at least one of the following modifications G61 {D,E}, N62{D,E}, N76{D,E}; *97aG, A98{G,S}, S99G, S101G, H120{N,V,Q,D}, P131{T,S}, Q137H, A194P, A228V, A230V, N261D. Or the protease variant comprises the following substitutions S9R, A15T, V68A, N218D and Q245R.

The parent subtilisin belongs to the subgroup I-S2.

In one embodiment, the parent subtilisin is a polypeptide comprising an amino acid sequence having at least 80% identity to SEQ ID NO: 23.

In one embodiment, the protease variant is a polypeptide sequence having at least 80% identity with SEQ ID NO: 24. In one embodiment the protease variant is a polypeptide sequence having at least 80% identity with SEQ ID NO: 25.

In a preferred embodiment, the protease is Blaze® sold by Novozymes A/S.

In a preferred embodiment of the invention, the dishwashing composition comprises enzymes capable of degrading cellulosic material and a protease and an amylase. The protease can be Blaze® and the amylase can be Stainzyme®.

The dishwashing composition may further comprise a surfactant. In addition other detergent components such as builders and polymers can be comprised in the dishwashing composition.

In one embodiment of the invention, the detergent composition reduces development of malodor. In one embodiment, the detergent composition reduces development of malodor in the dishwashing machine.

The dishwashing composition may further comprise a microorganism capable of degrading cellulosic material. A preferred microorganism is Bacillus subtilis SB3175 deposited at NRRL by Novozymes Biologicals Inc under deposition number NRRL B-50605.

Amino acid changes, as referenced above, may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for enzyme activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

The invention further concerns a dish washing method for automatic dishwashing comprising subjecting the dishes to an enzyme capable of degrading cellulosic material.

In order to facilitate the degradation of the cellulosic material, an aqueous solution of an acidic material can be used during the dishwashing process. The acidic material should be capable of lowering the pH to below 5. It is believed that when an acidic solution is used on cellulosic material, the structure of the cellulosic material opens up and is more susceptible to the enzymes capable of degrading cellulosic material. The process of degrading the cellulosic material is thereby faster than the degradation process without use of acid.

In one embodiment, the dishwashing method comprises the steps of:

a) Exposing the dishes to an aqueous solution of an acidic material (or alternatively: exposing the dishes to an aqueous solution having a pH below 5);

b) Exposing the dishes to a wash liquor comprising an enzyme preparation, which enzyme preparation comprises enzymes capable of degrading cellulosic material; and

c) Rinsing the dishes with water or an aqueous solution comprising a rinsing aid.

The one or more enzymes capable of degrading cellulosic material of step b) can be comprised the dishwashing composition of the present invention.

The sequence of the dishwashing steps can be varied. In one embodiment step a) is carried out before step b).

The exposing of the acidic material can be done a separate step in the dishwashing process, where the dishes are exposed to an aqueous solution of an acidic material before the dishes are exposed to washing liquor. This step can be performed in several ways. One way is by simply adding an acidic material to the interior of the dishwashing machine before starting the washing process. The acidic material will then dissolve when contacted with water. Another way is by circulating an aqueous solution of the acidic material before exposing the dishes to the wash liquor. Alternatively, the dishwashing composition is a powder or a granule and the acidic material is the outer layer of the powder granules. The acidic material is thereby released from the dishwashing composition and dissolved in the water in the dishwashing machine before the dishwashing composition is released. Another option is that the acidic material and the dishwashing composition are contained in a pouch having two or more compartments, where the acidic material is contained in one compartment and the dishwashing composition is contained in the other compartment. The compartment with the acidic material can then be released and dissolved before the dishwashing composition is released from the other compartment. Further, the composition can be a tablet having two or more layers, wherein the acidic material is the outer layer of the bar, which will then be released as described above.

In another embodiment of the invention, step a) of the dishwashing process is carried out simultaneously with step b). The acidic material can be part of the dishwashing composition and thereby be released and dissolved in the water at the same time as the dishwashing composition. The aqueous solution of the acidic material thereby forms part of the wash liquor. Alternatively the acidic material is added separately but simultaneously with the dishwashing composition to the chamber dedicated for the dishwashing composition. Thereby the acidic material will be release and dissolved at the same time as the dishwashing composition and form part of the wash liquor as described above.

In another embodiment of the invention, step a) of the dishwashing method is carried out simultaneously with step c). The acidic material can be part of the rinsing aid and thereby be released and dissolved in the water at the same time as the rinsing aid. The rinsing aid should be capable of lowering the pH below 4 during at least a period of the rinsing step. The pH may be even further lowered e.g. to below pH 3.5, such as below pH 3, below pH 2.5 or below pH 2. The period of lowering the pH may be at least 1 minute, such as at least 2 minutes, at least 3 minutes, at least 4 minutes, at least 5 minutes, at least 6 minutes or at least 7 minutes. The period of lowering the pH may even be as long as the time period for the full rinsing step.

The ability of lowering the pH during the rinsing step is due to a buffering agent. A buffer with strong buffer capacity at low pH, from pH 4 and below should be selected. The buffer capacity should correspond to the same effect as the pH drop was done with 15 ml 4M HCL/rinse cycle. The ability of lowering the pH during the rinsing step is due to a buffering agent selected from the group consisting of citric acid, acetic acid, potassium dihydrogen phosphate, boric acid, diethyl barbituric acid, Carmody buffer and Britton-Robinson buffer.

The rinsing aid is capable of lowering the pH of the water to below pH 5. In one embodiment the rinsing aid is capable of lowering the pH of the water to below 4.5, such as below pH 4, below pH 3.5 or below pH 3.

The dishwashing composition may be in the form of a powder, a bar, a homogenous tablet, a tablet having two or more layers, a pouch having one or more compartments, a regular or compact powder, a granule, a paste, a gel, or a regular, compact or concentrated liquid. The composition can be a powder or a granule where the acidic material is coated on the powder or granule as an outer layer. Alternatively the composition is a tablet having two or more layers, wherein the acidic material is the outer layer of the bar.

The composition can be a pouch having at least two compartments, wherein the acidic material is present in one compartment and is released before content of the other compartment(s).

The inventor has found that especially Cellic CTec3® is superior in degrading cellulosic material. When Cellic CTec3® is used in a dishwashing process as described above in combination with an aqueous solution of an acidic material, the degradation of cellulosic material is superior to degradation of cellulosic material in a similar process without the use of an acidic solution. Cellic CTec3® is an enzyme preparation comprising: Aspergillus fumigatus GH10 xylanase, Aspergillus fumigatus beta-xylosidase, Aspergillus fumigatus cellobiohydrolase I, Aspergillus fumigatus cellobiohydrolase II, Aspergillus fumigatus beta-glucosidase variant and Penicillium sp. (emersonii) GH61 polypeptide.

The one or more enzymes capable of degrading cellulosic material which is used in the dishwashing process can be comprised in a dishwashing composition according to the present invention.

The one or more enzymes capable of degrading cellulosic material can be used for degrading cellulosic material during a dish washing process. An acidic material may be used during the dishwashing process. The one or more enzymes capable of degrading cellulosic material can also be used for cleaning the interior of a dishwashing machine, e.g. cleaning of the drain where the cellulosic material remains after a washing process. The enzyme can be Cellic CTec3®.

The invention further concerns a method for cleaning the interior of an automated dish washing machine, which method comprises exposing the interior of the dish washing machine to one or more enzymes capable of degrading cellulosic material.

The one or more enzymes capable of degrading cellulosic material can be an enzyme preparation comprising:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

The Aspergillus fumigatus cellobiohydrolase I or homolog thereof of the enzyme preparation is selected from the group consisting of:
(v) a cellobiohydrolase I comprising or consisting of the mature polypeptide of SEQ ID NO: 2;
(vi) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
(vii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
(viii) a cellobiohydrolase I encoded by a polynucleotide that hybridizes under at least high stringency conditions, very high stringency conditions, with the mature polypeptide coding sequence of SEQ ID NO: 1 or the full-length complement thereof.

The Aspergillus fumigatus cellobiohydrolase II or homolog thereof is selected from the group consisting of:
(v) a cellobiohydrolase II comprising or consisting of the mature polypeptide of SEQ ID NO: 4;
(vi) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
(vii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
(viii) a cellobiohydrolase II encoded by a polynucleotide that hybridizes under at least high stringency conditions, very high stringency conditions, with the mature polypeptide coding sequence of SEQ ID NO: 3 or the full-length complement thereof.

The Aspergillus fumigatus beta-glucosidase or homolog thereof is selected from the group consisting of:
(vi) a beta-glucosidase comprising or consisting of the mature polypeptide of SEQ ID NO: 6;
(vii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
(viii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
(ix) a beta-glucosidase encoded by a polynucleotide that hybridizes under at least high stringency conditions, very high stringency conditions, with the mature polypeptide coding sequence of SEQ ID NO: 5 or the full-length complement thereof; and
(x) a beta-glucosidase variant comprising a substitution at one or more positions corresponding to positions 100, 283, 456, and 512 of the mature polypeptide of SEQ ID NO: 6, wherein the variant has beta-glucosidase activity; and

The Penicillium sp. GH61 polypeptide having cellulolytic enhancing activity or homolog thereof is selected from the group consisting of:
(v) a GH61 polypeptide having cellulolytic enhancing activity comprising or consisting of the mature polypeptide of SEQ ID NO: 6;
(vi) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
(vii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
(viii) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.

The beta-glucosidase variant of the enzyme preparation comprises one or more (several) substitutions selected from the group consisting of G142S, Q183R, H266Q, and D703G.

The enzyme preparation can further comprise one or more enzymes selected from the group consisting of:
(i) an Aspergillus fumigatus xylanase or homolog thereof,
(ii) an Aspergillus fumigatus beta-xylosidase or homolog thereof; or

a combination of (i) and (ii).

The Aspergillus fumigatus xylanase or homolog thereof is selected from the group consisting of:
(i) an Aspergillus fumigatus xylanase comprising or consisting of the mature polypeptide of SEQ ID NO: 10, SEQ ID NO: 12, or SEQ ID NO: 14;
(ii) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;
(iii) a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and
(iv) a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof.

The Aspergillus fumigatus beta-xylosidase or homolog thereof is selected from the group consisting of:
(i) beta-xylosidase comprising or consisting of the mature polypeptide of SEQ ID NO: 16;
(ii) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;
(iii) a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and
(iv) a beta-xylosidase encoded by a polynucleotide that hybridizes under at least high stringency conditions, very high stringency conditions, with the mature polypeptide coding sequence of SEQ ID NO: 15 or the full-length complement thereof.

In one embodiment of the invention, the dishwashing composition comprises a blend of an Aspergillus fumigatus GH10 xylanase and Aspergillus fumigatus beta-xylosidase with a Trichoderma reesei cellulase preparation containing Aspergillus fumigatus cellobiohydrolase I, Aspergillus fumigatus cellobiohydrolase II, Aspergillus fumigatus beta-glucosidase variant, and Penicillium sp. (emersonii) GH61 polypeptide.

In one embodiment of the invention, the dishwashing composition comprises a blend of an Aspergillus fumigatus GH10 xylanase and Aspergillus fumigatus beta-xylosidase with a Trichoderma reesei cellulase preparation containing Aspergillus fumigatus cellobiohydrolase I, Aspergillus fumigatus cellobiohydrolase II, Aspergillus fumigatus beta-glucosidase variant, and Penicillium sp. (emersonii) GH61 polypeptide, the Aspergillus fumigatus cellobiohydrolase I or homolog thereof, wherein the Aspergillus fumigatus cellobiohydrolase I or homolog thereof of the enzyme preparation is selected from the group consisting of:
(i) a cellobiohydrolase I comprising or consisting of the mature polypeptide of SEQ ID NO: 2;
(ii) a cellobiohydrolase I comprising or consisting of an amino acid sequence having at least 95%, at least 96%, at least 97%, at least 98%, or at least 99% sequence identity to the mature polypeptide of SEQ ID NO: 2; and

wherein the Aspergillus fumigatus cellobiohydrolase II or homolog thereof is selected from the group consisting of:
(i) a cellobiohydrolase II comprising or consisting of the mature polypeptide of SEQ ID NO: 4;
(ii) a cellobiohydrolase II comprising or consisting of an amino acid sequence having at least 95%, at least 96%, at least 97%, at least 98%, or at least 99% sequence identity to the mature polypeptide of SEQ ID NO: 4; and

wherein the Aspergillus fumigatus beta-glucosidase or homolog thereof is selected from the group consisting of:
(i) a beta-glucosidase comprising or consisting of the mature polypeptide of SEQ ID NO: 6;
(ii) a beta-glucosidase comprising or consisting of an amino acid sequence having at least 95%, at least 96%, at least 97%, at least 98%, or at least 99% sequence identity to the mature polypeptide of SEQ ID NO: 6; and

wherein the Penicillium sp. GH61 polypeptide having cellulolytic enhancing activity or homolog thereof is selected from the group consisting of:
(i) a GH61 polypeptide having cellulolytic enhancing activity comprising or consisting of the mature polypeptide of SEQ ID NO: 8;
(ii) a GH61 polypeptide having cellulolytic enhancing activity comprising or consisting of an amino acid sequence having at least 95%, at least 96%, at least 97%, at least 98%, or at least 99% sequence identity to the mature polypeptide of SEQ ID NO: 8; and

wherein the Aspergillus fumigatus xylanase or homolog thereof is selected from the group consisting of:
(i) an Aspergillus fumigatus xylanase comprising or consisting of the mature polypeptide of SEQ ID NO: 10, SEQ ID NO: 12, or SEQ ID NO: 14;
(ii) a xylanase comprising or consisting of an amino acid sequence having at least 95%, at least 96%, at least 97%, at least 98%, or at least 99% sequence identity to the mature polypeptide of SEQ ID NO: 10, SEQ ID NO: 12, or SEQ ID NO: 14; and

wherein the Aspergillus fumigatus beta-xylosidase or homolog thereof is selected from the group consisting of:
(i) beta-xylosidase comprising or consisting of the mature polypeptide of SEQ ID NO: 16;

a beta-xylosidase comprising or consisting of an amino acid sequence having at least 95%, at least 96%, at least 97%, at least 98%, or at least 99% sequence identity to the mature polypeptide of SEQ ID NO: 16. The one or more enzymes capable of degrading cellulosic material can be Cellic CTec3®. The enzymes capable of degrading cellulosic material can be comprised in the cleaning composition of the invention. The method may further comprise exposing the interior of the washing machine to an aqueous solution of an acidic material. The method for cleaning the interior of the dishwashing machine can be carried out at the same time as washing dishware in the dishwashing machine.

As described above, the exposing of the dishes to an aqueous solution of an acidic material in step a) of the dishwashing process can also be carried out at the same time as step c). The ability of lowering the pH during the rinsing step is due to a buffering agent. A buffer with strong buffer capacity at low pH, from pH 4 and below should be selected. The buffer capacity should correspond to the same effect as the pH drop was done with 15 ml 4M HCL/rinse cycle. The ability of lowering the pH during the rinsing step is due to a buffering agent selected from the group consisting of citric acid, acetic acid, potassium dihydrogen phosphate, boric acid, diethyl barbituric acid, Carmody buffer and Britton-Robinson buffer.

The present invention therefore also concerns a dishwashing rinsing aid, wherein the rinsing aid is capable of lowering the pH of the water to below pH 5. In one embodiment the rinsing aid is capable of lowering the pH of the water to below 4.5, such as below pH 4, below pH 3.5 or below pH 3.

The rinsing aid may comprise one or more enzymes capable of degrading cellulosic material. The enzymes capable of degrading cellulosic material can be Cellic CTec3®. The rinsing aid may be used in automated dishwashing.

### Concentration of the Enzyme of the Present Invention

In one embodiment of the present invention, the polypeptide of the present invention may be used in the dishwashing composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide of the present invention may also be incorporated in the detergent formulations disclosed in WO97/07202, which is hereby incorporated by reference.

### Surfactants

The dish washing composition can include at least one non-ionic surfactant. Suitable nonionic surfactants include, but are not limited to low-foaming nonionic (LFNI) surfactants. A LFNI surfactant is most typically used in an automatic dishwashing composition because of the improved water-sheeting action (especially from glassware) which they confer to the automatic dishwashing composition. They also may encompass non-silicone, phosphate or nonphosphate polymeric materials which are known to defoam food soils encountered in automatic dishwashing. The LFNI surfactant may have a relatively low cloud point and a high hydrophilic-lipophilic balance (HLB). Cloud points of 1% solutions in water are typically below about 32° C. and alternatively lower, e.g., 0° C., for optimum control of sudsing throughout a full range of water temperatures. If desired, a biodegradable LFNI surfactant having the above properties may be used.

A LFNI surfactant may include, but is not limited to: alkoxylated surfactants, especially ethoxylates derived from primary alcohols, and blends thereof with more sophisticated surfactants, such as the polyoxypropylene/polyoxyethylene/polyoxypropylene reverse block polymers. Suitable block polyoxyethylene-polyoxypropylene polymeric compounds that meet the requirements may include those based on ethylene glycol, propylene glycol, glycerol, trimethylolpropane and ethylenediamine, and mixtures thereof. Polymeric compounds made from a sequential ethoxylation and propoxylation of initiator compounds with a single reactive hydrogen atom, such as C 12-is aliphatic alcohols, do not generally provide satisfactory suds control in Automatic dishwashing compositions. However, certain of the block polymer surfactant compounds designated as PLURONIC® and TETRONIC® by the BASF-Wyandotte Corp., Wyandotte, Mich., are suitable in Automatic dishwashing compositions. The LFNI surfactant can optionally include a propylene oxide in an amount up to about 15% by weight. Other LFNI surfactants can be prepared by the processes described in U.S. Pat. No. 4,223,163. The LFNI surfactant may also be derived from a straight chain fatty alcohol containing from about 16 to about 20 carbon atoms (C16-C20 alcohol), alternatively a Ci8 alcohol, condensed with an average of from about 6 to about 15 moles, or from about 7 to about 12 moles, and alternatively, from about 7 to about 9 moles of ethylene oxide per mole of alcohol. The ethoxylated nonionic surfactant so derived may have a narrow ethoxylate distribution relative to the average.

In certain embodiments, a LFNI surfactant having a cloud point below 30° C. may be present in an amount from about 0.01% to about 60%, or from about 0.5% to about 10% by weight, and alternatively, from about 1% to about 5% by weight of the composition

In preferred embodiments, the surfactant is a non-ionic surfactant or a non-ionic surfactant system having a phase inversion temperature, as measured at a concentration of 1% in distilled water, between 40 and 70° C., preferably between 45 and 65° C. By a “non-ionic surfactant system” is meant herein a mixture of two or more non-ionic surfactants. Preferred for use herein are non-ionic surfactant systems. They seem to have improved cleaning and finishing properties and stability in product than single non-ionic surfactants. Suitable nonionic surfactants include: i) ethoxylated non-ionic surfactants prepared by the reaction of a monohydroxy alkanol or alkyphenol with 6 to 20 carbon atoms with preferably at least 12 moles particularly preferred at least 16 moles, and still more preferred at least 20 moles of ethylene oxide per mole of alcohol or alkylphenol; ii) alcohol alkoxylated surfactants having a from 6 to 20 carbon atoms and at least one ethoxy and propoxy group. Preferred for use herein are mixtures of surfactants i) and ii).

Another suitable non-ionic surfactants are epoxy-capped poly(oxyalkylated) alcohols represented by the formula:

R₁O[CH₂CH(CH₃)O]ₓ[CH₂CH₂O]_{y}[CH₂CH(OH)R₂] (I)

wherein R₁ is a linear or branched, aliphatic hydrocarbon radical having from 4 to 18 carbon atoms; R₂ is a linear or branched aliphatic hydrocarbon radical having from 2 to 26 carbon atoms; x is an integer having an average value of from 0.5 to 1.5, more preferably about 1; and y is an integer having a value of at least 15, more preferably at least 20. Preferably, the surfactant of formula I has at least about 10 carbon atoms in the terminal epoxide unit [CH₂CH(OH)R₂]. Suitable surfactants of formula I are Olin Corporation's POLY-TERGENT® SLF-18B nonionic surfactants, as described, for example, in WO 94/22800, published Oct. 13, 1994 by Olin Corporation.

Preferably non-ionic surfactants and/or system herein have a Draves wetting time of less than 360 seconds, preferably less than 200 seconds, more preferably less than 100 seconds and especially less than 60 seconds as measured by the Draves wetting method (standard method ISO 8022 using the following conditions; 3-g hook, 5-g cotton skein, 0.1% by weight aqueous solution at a temperature of 25° C.). Amine oxides surfactants are also useful in the present invention as anti-redeposition surfactants include linear and branched compounds having the formula:

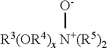

wherein R³ is selected from an alkyl, hydroxyalkyl, acylamidopropoyl and alkyl phenyl group, or mixtures thereof, containing from 8 to 26 carbon atoms, preferably 8 to 18 carbon atoms; R⁴ is an alkylene or hydroxyalkylene group containing from 2 to 3 carbon atoms, preferably 2 carbon atoms, or mixtures thereof; x is from 0 to 5, preferably from 0 to 3; and each R⁵ is an alkyl or hydroxyalkyl group containing from 1 to 3, preferably from 1 to 2 carbon atoms, or a polyethylene oxide group containing from 1 to 3, preferable 1, ethylene oxide groups. The R⁵ groups can be attached to each other, e.g., through an oxygen or nitrogen atom, to form a ring structure.

These amine oxide surfactants in particular include C₁₀-C₁₈ alkyl dimethyl amine oxides and C₈-C₁₈ alkoxy ethyl dihydroxyethyl amine oxides. Examples of such materials include dimethyloctylamine oxide, diethyldecylamine oxide, bis-(2-hydroxyethyl)dodecylamine oxide, dimethyldodecylamine oxide, dipropyltetradecylamine oxide, methylethylhexadecylamine oxide, dodecylamidopropyl dimethylamine oxide, cetyl dimethylamine oxide, stearyl dimethylamine oxide, tallow dimethylamine oxide and dimethyl-2-hydroxyoctadecylamine oxide. Preferred are C₁₀-C₁₈ alkyl dimethylamine oxide, and C₁₀-C₁₈ acylamido alkyl dimethylamine oxide. Surfactants and especially non-ionic surfactants may be present in amounts from 0 to 10% by weight, preferably from 0.1% to 10%, and most preferably from 0.25% to 6%.

### Sulfonated Polymer

The polymer, if used, is used in any suitable amount from about 0.1% to about 20%, preferably from 1% to about 15%, more preferably from 2% to 10% by weight of the composition. Sulfonated/carboxylated polymers are particularly suitable for the compositions contained in the pouch of the invention.

Suitable sulfonated/carboxylated polymers described herein may have a weight average molecular weight of less than or equal to about 100,000 Da, or less than or equal to about 75,000 Da, or less than or equal to about 50,000 Da, or from about 3,000 Da to about 50,000, preferably from about 5,000 Da to about 45,000 Da.

As noted herein, the sulfonated/carboxylated polymers may comprise (a) at least one structural unit derived from at least one carboxylic acid monomer having the general formula (I):

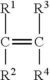

wherein R¹ to R⁴ are independently hydrogen, methyl, carboxylic acid group or CH₂COOH and wherein the carboxylic acid groups can be neutralized; (b) optionally, one or more structural units derived from at least one nonionic monomer having the general formula (II):

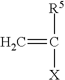

wherein R⁵ i is hydrogen, C₁ to C₆ alkyl, or C₁ to C₆ hydroxyalkyl, and X is either aromatic (with R⁵ being hydrogen or methyl when X is aromatic) or X is of the general formula (III):

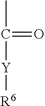

wherein R⁶ is (independently of R⁵) hydrogen, C₁ to C₆ alkyl, or C₁ to C₆ hydroxyalkyl, and Y is 0 or N; and at least one structural unit derived from at least one sulfonic acid monomer having the general formula (IV):

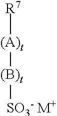

wherein R⁷ is a group comprising at least one sp² bond, A is O, N, P, S or an amido or ester linkage, B is a mono- or polycyclic aromatic group or an aliphatic group, each t is independently 0 or 1, and M⁺ is a cation. In one aspect, R⁷ is a C₂ to C₆ alkene. In another aspect, R⁷ is ethene, butene or propene.

Preferred carboxylic acid monomers include one or more of the following: acrylic acid, maleic acid, itaconic acid, methacrylic acid, or ethoxylate esters of acrylic acids, acrylic and methacrylic acids being more preferred. Preferred sulfonated monomers include one or more of the following: sodium (meth) allyl sulfonate, vinyl sulfonate, sodium phenyl (meth) allyl ether sulfonate, or 2-acrylamido-methyl propane sulfonic acid. Preferred non-ionic monomers include one or more of the following: methyl (meth) acrylate, ethyl (meth) acrylate, t-butyl (meth) acrylate, methyl (meth) acrylamide, ethyl (meth) acrylamide, t-butyl (meth) acrylamide, styrene, or [alpha]-methyl styrene.

Preferably, the polymer comprises the following levels of monomers: from about 40 to about 90%, preferably from about 60 to about 90% by weight of the polymer of one or more carboxylic acid monomer; from about 5 to about 50%, preferably from about 10 to about 40% by weight of the polymer of one or more sulfonic acid monomer; and optionally from about 1% to about 30%, preferably from about 2 to about 20% by weight of the polymer of one or more non-ionic monomer. An especially preferred polymer comprises about 70% to about 80% by weight of the polymer of at least one carboxylic acid monomer and from about 20% to about 30% by weight of the polymer of at least one sulfonic acid monomer. 99 The carboxylic acid is preferably (meth)acrylic acid. The sulfonic acid monomer is preferably one of the following: 2-acrylamido methyl-1-propanesulfonic acid, 2-methacrylamido-2-methyl-1-propanesulfonic acid, 3-methacrylamido-2-hydroxypropanesulfonic acid, allysulfonic acid, methallysulfonic acid, allyloxybenzenesulfonic acid, methallyloxybenzensulfonic acid, 2-hydroxy-3-(2-propenyloxy)propanesulfonic acid, 2-methyl-2-propene-1-sulfonic acid, styrene sulfonic acid, vinylsulfonic acid, 3-sulfopropyl acrylate, 3-sulfopropyl methacrylate, sulfomethylacrylamid, sulfomethylmethacrylamide, and water soluble salts thereof. The unsaturated sulfonic acid monomer is most preferably 2-acrylamido-2-propanesulfonic acid (AMPS).

Preferred commercial available polymers include: Alcosperse 240, Aquatreat AR 540 and Aquatreat MPS supplied by Alco Chemical; Acumer 3100, Acumer 2000, Acusol 587G and Acusol 588G supplied by Rohm & Haas; Goodrich K-798, K-775 and K-797 supplied by BF Goodrich; and ACP 1042 supplied by ISP technologies Inc. Particularly preferred polymers are Acusol 587G and Acusol 588G supplied by Rohm & Haas.

In the polymers, all or some of the carboxylic or sulfonic acid groups can be present in neutralized form, i.e. the acidic hydrogen atom of the carboxylic and/or sulfonic acid group in some or all acid groups can be replaced with metal ions, preferably alkali metal ions and in particular with sodium ions.

Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in ADW detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

Inorganic and organic bleaches are suitable cleaning actives for use herein. Inorganic bleaches include perhydrate salts such as perborate, percarbonate, perphosphate, persulfate and persilicate salts. The inorganic perhydrate salts are normally the alkali metal salts. The inorganic perhydrate salt may be included as the crystalline solid without additional protection. Alternatively, the salt can be coated.

Alkali metal percarbonates, particularly sodium percarbonate are preferred perhydrates for use herein. The percarbonate is most preferably incorporated into the products in a coated form which provides in-product stability. A suitable coating material providing in product stability comprises mixed salt of a water-soluble alkali metal sulphate and carbonate. Such coatings together with coating processes have previously been described in GB-1,466,799. The weight ratio of the mixed salt coating material to percarbonate lies in the range from 1:200 to 1:4, more preferably from 1:99 to 1 9, and most preferably from 1:49 to 1:19. Preferably, the mixed salt is of sodium sulphate and sodium carbonate which has the general formula Na2SO4.n.Na2CO3 wherein n is from 0.1 to 3, preferably n is from 0.3 to 1.0 and most preferably n is from 0.2 to 0.5.

Another suitable coating material providing in product stability, comprises sodium silicate of SiO2: Na2O ratio from 1.8:1 to 3.0:1, preferably L8:I to 2.4:1, and/or sodium metasilicate, preferably applied at a level of from 2% to 10%, (normally from 3% to 5%) of SiO2 by weight of the inorganic perhydrate salt. Magnesium silicate can also be included in the coating. Coatings that contain silicate and borate salts or boric acids or other inorganics are also suitable.

Other coatings which contain waxes, oils, fatty soaps can also be used advantageously within the present invention.

Potassium peroxymonopersulfate is another inorganic perhydrate salt of utility herein. Typical organic bleaches are organic peroxyacids including diacyl and tetraacylperoxides, especially diperoxydodecanedioc acid, diperoxytetradecanedioc acid, and diperoxyhexadecanedioc acid. Dibenzoyl peroxide is a preferred organic peroxyacid herein. Mono- and diperazelaic acid, mono- and diperbrassylic acid, and Nphthaloylaminoperoxicaproic acid are also suitable herein. The diacyl peroxide, especially dibenzoyl peroxide, should preferably be present in the form of particles having a weight average diameter of from about 0.1 to about 100 microns, preferably from about 0.5 to about 30 microns, more preferably from about 1 to about 10 microns. Preferably, at least about 25%, more preferably at least about 50%, even more preferably at least about 75%, most preferably at least about 90%, of the particles are smaller than 10 microns, preferably smaller than 6 microns. Diacyl peroxides within the above particle size range have also been found to provide better stain removal especially from plastic dishware, while minimizing undesirable deposition and filming during use in automatic dishwashing machines, than larger diacyl peroxide particles. The preferred diacyl peroxide particle size thus allows the formulator to obtain good stain removal with a low level of diacyl peroxide, which reduces deposition and filming. Conversely, as diacyl peroxide particle size increases, more diacyl peroxide is needed for good stain removal, which increases deposition on surfaces encountered during the dishwashing process. Further typical organic bleaches include the peroxy acids, particular examples being the alkylperoxy acids and the arylperoxy acids. Preferred representatives are (a) peroxybenzoic acid and its ring-substituted derivatives, such as alkylperoxybenzoic acids, but also peroxy-[alpha]-naphthoic acid and magnesium monoperphthalate, (b) the aliphatic or substituted aliphatic peroxy acids, such as peroxylauric acid, peroxystearic acid, [epsilon]-phthalimidoperoxycaproic acid[phthaloiminoperoxyhexanoic acid (PAP)], o-carboxybenzamidoperoxycaproic acid, N-nonenylamidoperadipic acid and N-nonenylamidopersuccinates, and (c) aliphatic and araliphatic peroxydicarboxylic acids, such as 1,12-diperoxycarboxylic acid, 1,9-diperoxyazelaic acid, diperoxysebacic acid, diperoxybrassylic acid, the diperoxyphthalic acids, 2-decyldiperoxybutane-1,4-dioic acid, N,N-terephthaloyldi(6-aminopercaproic acid).

### Bleach Activators

Bleach activators are typically organic peracid precursors that enhance the bleaching action in the course of cleaning at temperatures of 60[deg.] C and below. Bleach activators suitable for use herein include compounds which, under perhydrolysis conditions, give aliphatic peroxoycarboxylic acids having preferably from 1 to 10 carbon atoms, in particular from 2 to 4 carbon atoms, and/or optionally substituted perbenzoic acid. Suitable substances bear O-acyl and/or N-acyl groups of the number of carbon atoms specified and/or optionally substituted benzoyl groups. Preference is given to polyacylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl- or isononanoyloxybenzenesulfonate (n- or iso-NOBS), carboxylic anhydrides, in particular phthalic anhydride, acylated polyhydric alcohols, in particular triacetin, ethylene glycol diacetate and 2,5-diacetoxy-2,5-dihydrofuran and also triethylacetyl citrate (TEAC). Bleach activators if included in the compositions of the invention are in a level of from about 0.1 to about 10%, preferably from about 0.5 to about 2% by weight of the composition.

### Bleach Catalysts

Bleach catalysts preferred for use herein include the manganese triazacyclononane, MnTACN and related complexes (U.S. Pat. No. 4,246,612, U.S. Pat. No. 5,227,084); Co, Cu, Mn and Fe bispyridylamine and related complexes (U.S. Pat. No. 5,114,611); and pentamine acetate cobalt(III) and related complexes (U.S. Pat. No. 4,810,410). A complete description of bleach catalysts suitable for use herein can be found in WO 99/06521, pages 34, line 26 to page 40, line 16. Bleach catalyst if included in the compositions of the invention are in a level of from about 0.1 to about 10%, preferably from about 0.5 to about 2% by weight of the composition. Oxidoreductases, for example oxidases, oxygenases, catalases, peroxidases such as halo-, chloro-, bromo-, lignin, glucose, or manganese peroxidases, dioxygenases, or laccases (phenoloxidases, polyphenoloxidases), can also be used according to the present invention to intensify the bleaching effect. Advantageously, preferably organic, particularly preferably aromatic compounds that interact with the enzymes are additionally added in order to enhance the activity of the relevant oxidoreductases (enhancers) or, if there is a large difference in redox potentials between the oxidizing enzymes and the stains, to ensure electron flow (mediators).

### Silicates

Preferred silicates are sodium silicates such as sodium disilicate, sodium metasilicate and crystalline phyllosilicates. Silicates if present are at a level of from about 1 to about 20%, preferably from about 5 to about 15% by weight of composition.

### Metal Care Agents

Metal care agents may prevent or reduce the tarnishing, corrosion or oxidation of metals, including aluminium, stainless steel and non-ferrous metals, such as silver and copper. Suitable examples include one or more of the following:

(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups and hydroxyl, thio, phenyl or halogen such as fluorine, chlorine, bromine and iodine.

(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K̂TiF6, K̂ZrF6, CoSO4, Co(NOs)2 and Ce(NOs)3, zinc salts, for example zinc sulphate, hydrozincite or zinc acetate; (c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties.

Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Enzymes

The detergent additive as well as the detergent composition may comprise one or more [additional] enzymes such as a protease, lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

### Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V1041,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Savinase® is marketed by NOVOZYMES NS. It is subtilisin 309 from B. Lentus and differs from BAALKP only in one position (N87S). Savinase® has the amino acid sequence SEQ ID NO: 18.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases which can be used together with the enzyme preparation of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase and Preferenz S100 (from Genencor International Inc./DuPont).

### Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in ADW detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use ADW detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

The invention is further summarized in the following paragraphs:
1. A dishwashing composition comprising a builder and one or more enzymes capable of degrading cellulosic material, wherein the one or more enzymes capable of degrading cellulosic material is an enzyme preparation comprising:
   a) a cellobiohydrolase I;
   b) a cellobiohydrolase II;
   c) a beta-glucosidase or variant thereof; and
   d) a. GH61 polypeptide having cellulolytic enhancing activity; or homologs thereof.
2. Dishwashing composition according to paragraph 1, wherein the one or more enzymes capable of degrading cellulosic material is an enzyme preparation comprising:
   (i) an Aspergillus fumigatus cellobiohydrolase I;
   (ii) an Aspergillus fumigatus cellobiohydrolase II;
   (iii) an Aspergillus fumigatus beta-glucosidase or variant thereof; and
   (iv) a Penicillium sp. GH61 polypeptide having cellulolytic enhancing activity; or homologs thereof.
3. Dishwashing composition Dishwashing composition according to any of the preceding paragraphs, wherein the Aspergillus fumigatus cellobiohydrolase I or homolog thereof of the enzyme preparation is selected from the group consisting of:
   (i) a cellobiohydrolase I comprising or consisting of the mature polypeptide of SEQ ID NO: 2;
   (ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
   (iii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
   (iv) a cellobiohydrolase I encoded by a polynucleotide that hybridizes under at least high stringency conditions, very high stringency conditions, with the mature polypeptide coding sequence of SEQ ID NO: 1 or the full-length complement thereof;
wherein the Aspergillus fumigatus cellobiohydrolase II or homolog thereof is selected from the group consisting of:
   (i) a cellobiohydrolase II comprising or consisting of the mature polypeptide of SEQ ID NO: 4;
   (ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
   (iii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
   (iv) a cellobiohydrolase II encoded by a polynucleotide that hybridizes under at least high stringency conditions, very high stringency conditions, with the mature polypeptide coding sequence of SEQ ID NO: 3 or the full-length complement thereof;
wherein the Aspergillus fumigatus beta-glucosidase or homolog thereof is selected from the group consisting of:
   (i) a beta-glucosidase comprising or consisting of the mature polypeptide of SEQ ID NO: 6;
   (ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
   (iii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
   (iv) a beta-glucosidase encoded by a polynucleotide that hybridizes under at least high stringency conditions, very high stringency conditions, with the mature polypeptide coding sequence of SEQ ID NO: 5 or the full-length complement thereof; and
   (v) a beta-glucosidase variant comprising a substitution at one or more positions corresponding to positions 100, 283, 456, and 512 of the mature polypeptide of SEQ ID NO: 6, wherein the variant has beta-glucosidase activity; and
wherein the Penicillium sp. GH61 polypeptide having cellulolytic enhancing activity or homolog thereof is selected from the group consisting of:
   (i) a GH61 polypeptide having cellulolytic enhancing activity comprising or consisting of the mature polypeptide of SEQ ID NO: 8;
   (ii) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
   (iii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
   (iv) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.
4. Dishwashing composition according to any of the preceding paragraphs, wherein the beta-glucosidase variant of the enzyme preparation comprises one or more (several) substitutions selected from the group consisting of G142S, Q183R, H266Q, and D703G.
5. Dishwashing composition according to any of the preceding paragraphs, wherein the enzyme preparation further comprises one or more enzymes selected from the group consisting of:
   (i) an Aspergillus fumigatus xylanase or homolog thereof,
   (ii) an Aspergillus fumigatus beta-xylosidase or homolog thereof; or
   (iii) a combination of (i) and (ii);
wherein the Aspergillus fumigatus xylanase or homolog thereof is selected from the group consisting of:
   (i) an Aspergillus fumigatus xylanase comprising or consisting of the mature polypeptide of SEQ ID NO: 10, SEQ ID NO: 12, or SEQ ID NO: 14;
   (ii) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;
   (iii) a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and
   (iv) a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof; and
wherein the Aspergillus fumigatus beta-xylosidase or homolog thereof is selected from the group consisting of:
   (i) beta-xylosidase comprising or consisting of the mature polypeptide of SEQ ID NO: 16;
   (ii) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;
   (iii) a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and
   (iv) a beta-xylosidase encoded by a polynucleotide that hybridizes under at least high stringency conditions, very high stringency conditions, with the mature polypeptide coding sequence of SEQ ID NO: 15 or the full-length complement thereof.
6. Dishwashing composition according to any of the preceding paragraphs, wherein the dishwashing composition comprises at least one enzyme in addition to the enzymes in the enzyme preparation.
7. Dishwashing composition according to any of the preceding paragraphs, wherein the composition comprises amylase and/or protease.
8. Dishwashing composition according to any of the preceding paragraphs, wherein the amylase is an alpha-amylase or a glucoamylase.
9. Dishwashing composition according to any of the preceding paragraphs, wherein the amylase is of bacterial or fungal origin.
10. Dishwashing composition according to any of the preceding paragraphs, wherein the amylase is an alpha-amylase obtained from Bacillus, such as Bacillus licheniformis.
11. Dishwashing composition according to any of the preceding paragraphs, wherein the amylase is selected from the group consisting of:
   (i) a polypeptide having at least 90%, such as at least 95%, sequence identity to SEQ ID NO: 17;
   (ii) a polypeptide having at least 90%, such as at least 95%, sequence identity to SEQ ID NO: 17 or a variant thereof wherein the polypeptide comprises a substitution, a deletion or an insertion in one of more of positions: 181, 182, 183, 184, 195, 206, 212, 216 and/or 269;
   (iii) a polypeptide having at least 90%, such as at least 95%, sequence identity to SEQ ID NO: 18, SEQ ID NO: 19 or SEQ ID NO: 20;
   (iv) a polypeptide having at least 90%, such as at least 95%, sequence identity to SEQ ID NO: 18, SEQ ID NO: 19 or SEQ ID NO: 20 or a variant thereof wherein the polypeptide comprises a substitution, a deletion or an insertion in one of more of positions: 140, 183, 184 195, 206, 243, 260, 304 and/or 476;
12. Dishwashing composition according to any of the preceding paragraphs, wherein the amylase is an alpha-amylase having SEQ ID NO: 17 or a variant thereof having at least 80%, at least 85% or at least 90% sequence identity to SEQ ID NO: 17 and having a substitution, a deletion or an insertion of one amino acids downstream for the amino acid corresponding to the positions in the amylase having SEQ ID NO: 17: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484; particular preferred amylases include such a variant having a deletion of D183 and G184 and having the substitutions R118K, N195F, R320K and R458K and a variant additionally having substitutions in one or more positions selected from the group: M9, G149, G182, G186, M202, T257, Y295, N299, M323, A339 and E345, most preferred a variant additionally having substitutions in all these positions; or a variant alpha-amylase derived from a parent α-amylase derived from B. licheniformis comprising the mutation:
A1*+N2*+L3V+M15T+R23K+S29A+A30E+Y31H+A33S+E34D+H35I+M197T.
13. Dishwashing composition according to any of the preceding paragraphs, wherein the protease is chemically modified or protein engineered.
14. Dishwashing composition according to any of the preceding paragraphs, wherein the protease is a serine protease or a metalloprotease, preferably an alkaline microbial protease or a trypsin-like protease.
15. Dishwashing composition according to any of the preceding paragraphs, wherein the is selected from the group consisting of Bacillus, e.g., subtilisin Novo, subtilisin Carlsberg, subtilisin 309, subtilisin 147, subtilisin 168, trypsin of bovine origin, trypsin of porcine origin and Fusarium protease.
16. Dishwashing composition according to any of the preceding paragraphs, wherein the protease has at least 90%, such as at least 95%, sequence identity to SEQ ID NO: 21.
17. Dishwashing composition according to any of the preceding paragraphs, wherein the protease has at least 90% identity to the amino acid sequence of SEQ ID NO: 21 or a variant thereof with substitutions in one or more of the following positions: 27, 36, 57, 76, 87, 97, 101, 104, 120, 123, 167, 170, 194, 206, 218, 222, 224, 235, and 274, preferably the variant is an alkaline protease having at least 90% identity to the amino acid sequence of SEQ ID NO: 21 with the following substitution: M222S or substitutions N76D+G195E.
18. Dishwashing composition according to any of the preceding paragraphs, wherein the protease is a subtilisin variant, wherein the variant comprises the substitutions 9R, 15T, 68A, 245R and 218 {D,G,V} in a parent subtilisin, and wherein the positions corresponds to the positions of the mature polypeptide of SEQ ID NO:22 [BPN′].
19. Dishwashing composition according to paragraph 18, wherein the substitution at position 218 is with D.
20. Dishwashing composition according to any of paragraphs 18-19, wherein the variant further comprises at least one of the following modifications G61 {D,E}, N62{D,E}, N76{D,E}; *97aG, A98{G,S}, S99G, S101G, H120{N,V,Q,D}, P131{T,S}, Q137H, A194P, A228V, A230V, N261 D.
21. Dishwashing composition according to any of paragraphs 18-20, wherein the variant comprises the following substitutions S9R, A15T, V68A, N218D and Q245R.
22. Dishwashing composition according to any of paragraphs 18-21, wherein the parent subtilisin belongs to the subgroup I-S2.
23. Dishwashing composition according to any of paragraphs 1-15, wherein the parent subtilisin is a polypeptide comprising an amino acid sequence having at least 80% identity to SEQ ID NO: 23.
24. Dishwashing composition according to any of paragraphs 1-15, wherein the variant is a polypeptide sequence having at least 80% identity with SEQ ID NO: 24.
25. Dishwashing composition according to any of paragraphs 1-15, wherein the variant is a polypeptide sequence having at least 80% identity with SEQ ID NO: 25.
26. Dishwashing composition according to any of the preceding paragraphs, wherein the composition further comprises a surfactant.
27. Dishwashing composition according to any of the preceding paragraphs, wherein the composition further comprises one or more builders and one or more polymer.
28. Dishwashing composition according to any of the preceding composition paragraphs, wherein the composition further comprises one or more components selected from the group consisting of polymers, bleaching systems, bleach activators, bleach catalysts, silicates, dyestuff and metal care agents.
29. Dishwashing composition according to any of the preceding paragraphs, wherein the composition further comprises an acidic material.
30. Dishwashing composition according to any of the preceding paragraphs, wherein the acidic material is selected from the group consisting of citric acid, acetic acid, potassium dihydrogen phosphate, boric acid, diethyl barbituric acid, Carmody buffer and Britton-Robinson buffer
31. Dishwashing composition according to any of the preceding paragraphs, wherein the composition is in the form of a powder, a bar, a homogenous tablet, a tablet having two or more layers, a pouch having one or more compartments, a regular or compact powder, a granule, a paste, a gel, or a regular, compact or concentrated liquid.
32. Dishwashing composition according to any of the preceding paragraphs, wherein the composition is a powder or a granule and the acidic material is the outer layer of the powder granules.
33. Dishwashing composition according to any of the preceding paragraphs, wherein the composition is a tablet having two or more layers, wherein the acidic material is the outer layer of the bar.
34. Dishwashing composition according to any of the preceding paragraphs, wherein the composition is a pouch having at least two compartments, wherein the acidic material is present in one compartment and is released before content of the other compartment(s).
35. Dishwashing composition according to any of the preceding paragraphs, wherein the composition is for use in ADW.
36. Dishwashing composition according to any of the preceding paragraphs, wherein the composition further comprises a microorganism capable of degrading cellulosic material.
37. Dishwashing composition according to paragraph 36, wherein the microorganism is Bacillus subtilis SB3175.
38. A washing method comprising exposing the dish ware to a wash liquor comprising the dishwashing composition according to any of paragraphs 1-37.
39. Method according to paragraph 38, wherein the method comprises the steps of:
   (i) Exposing the dishes to an aqueous solution having a pH below 4;
   (ii) Exposing the dishes to a wash liquor comprising the dishwashing composition according to any of paragraphs 1-37; and
   (iii) Rinsing the dishes with water or an aqueous solution comprising a rinsing aid;
   wherein step a) is carried out before step b) or step a) is carried out simultaneously with step b) or step c).
40. Method according to any of the preceding method paragraphs, wherein step a is carried out by adding an acidic material to interior of a dishwasher before the dishwasher is started and the aqueous solution of the acidic material in step a) is obtained by starting the dishwasher and thereby exposing the acidic material to water.
41. Method according to any of the preceding method paragraphs, wherein step a) and step b) is carried out simultaneously by simultaneous use of a dishwashing composition and an acidic material or by use of an dishwashing composition comprising an acidic material.
42. Method according to any of the preceding method paragraphs, wherein step a) and step c) is carried out simultaneously by simultaneous use of an acidic material and water for rinsing or by use of an aqueous solution comprising a rinsing aid with an acidic material.
43. Method according to any of the preceding method paragraphs, wherein the washing method is hand dishwashing or ADW.
44. Use of the dishwashing composition according to any of paragraphs 1-37 in a dish washing process.
45. Use according to paragraph 44 for washing or cleaning of dishware.
46. Use according to any of the preceding use paragraphs for cleaning the interior of a dishwashing machine such as walls, baskets, nozzles, pumps, sump, filters, pipelines, drains, and outlets.
47. Use according to any of the preceding use paragraphs comprising the use of an acidic material.
48. Use according to any of the preceding use paragraphs for degrading cellulosic material on dishware in an automated dish washing machine.
49. A cleaning method for cleaning the interior of an automated dish washing machine, which method comprises exposing the interior of the dish washing machine to the dishwashing composition according to any of paragraphs 1-37.
50. Cleaning method according to paragraph 49, wherein the method comprises exposing the interior of the washing machine to an aqueous solution of an acidic material.
51. Cleaning method according to any of paragraphs 49-50, wherein the method is carried out at the same time as washing dishware in the dishwashing machine.
52. A rinsing aid for use in an automatic dish washing method, wherein the rinsing aid is capable of lowering the pH below 4 during at least a period of a rinse cycle in an automated dishwashing process.
53. Rinsing aid according to paragraph 52, wherein the pH is below pH 3.5, such as below pH 3, below pH 2.5 or below pH 2.
54. Rinsing aid according to any of paragraphs 52-53, wherein the aid comprises an enzyme preparation, which enzyme preparation comprises enzymes capable of degrading cellulosic material.
55. Rinsing aid according to any of paragraphs 52-54, wherein the enzyme preparation comprises:
   (i) an Aspergillus fumigatus cellobiohydrolase I;
   (ii) an Aspergillus fumigatus cellobiohydrolase II;
   (iii) an Aspergillus fumigatus beta-glucosidase or variant thereof; and
   (iv) a Penicillium sp. GH61 polypeptide having cellulolytic enhancing activity; or homologs thereof.
56. Rinsing aid according to any of paragraphs 52-55, wherein the Aspergillus fumigatus cellobiohydrolase I or homolog thereof of the enzyme preparation is selected from the group consisting of:
   (i) a cellobiohydrolase I comprising or consisting of the mature polypeptide of SEQ ID NO: 2;
   (ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
   (iii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
   (iv) a cellobiohydrolase I encoded by a polynucleotide that hybridizes under at least high stringency conditions, very high stringency conditions, with the mature polypeptide coding sequence of SEQ ID NO: 1 or the full-length complement thereof;
wherein the Aspergillus fumigatus cellobiohydrolase II or homolog thereof is selected from the group consisting of:
   (i) a cellobiohydrolase II comprising or consisting of the mature polypeptide of SEQ ID NO: 4;
   (ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
   (iii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
   (iv) a cellobiohydrolase II encoded by a polynucleotide that hybridizes under at least high stringency conditions, very high stringency conditions, with the mature polypeptide coding sequence of SEQ ID NO: 3 or the full-length complement thereof;
wherein the Aspergillus fumigatus beta-glucosidase or homolog thereof is selected from the group consisting of:
   (i) a beta-glucosidase comprising or consisting of the mature polypeptide of SEQ ID NO: 6;
   (ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
   (iii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
   (iv) a beta-glucosidase encoded by a polynucleotide that hybridizes under at least high stringency conditions, very high stringency conditions, with the mature polypeptide coding sequence of SEQ ID NO: 5 or the full-length complement thereof; and
   (v) a beta-glucosidase variant comprising a substitution at one or more positions corresponding to positions 100, 283, 456, and 512 of the mature polypeptide of SEQ ID NO: 6, wherein the variant has beta-glucosidase activity; and
wherein the Penicillium sp. GH61 polypeptide having cellulolytic enhancing activity or homolog thereof is selected from the group consisting of:
   (i) a GH61 polypeptide having cellulolytic enhancing activity comprising or consisting of the mature polypeptide of SEQ ID NO: 6;
   (ii) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
   (iii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
   (iv) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.
57. Rinsing aid according to any of paragraphs 52-56, wherein the beta-glucosidase variant of the enzyme preparation comprises one or more (several) substitutions selected from the group consisting of G142S, Q183R, H266Q, and D703G.
58. Rinsing aid according to any of paragraphs 52-57, wherein the enzyme preparation further comprises one or more enzymes selected from the group consisting of:
   (i) an Aspergillus fumigatus xylanase or homolog thereof,
   (ii) an Aspergillus fumigatus beta-xylosidase or homolog thereof; or
   (iii) a combination of (i) and (ii);
wherein the Aspergillus fumigatus xylanase or homolog thereof is selected from the group consisting of:
   (i) an Aspergillus fumigatus xylanase comprising or consisting of the mature polypeptide of SEQ ID NO: 10, SEQ ID NO: 12, or SEQ ID NO: 14;
   (ii) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;
   (iii) a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and
   (iv) a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof; and
wherein the Aspergillus fumigatus beta-xylosidase or homolog thereof is selected from the group consisting of:
   (i) beta-xylosidase comprising or consisting of the mature polypeptide of SEQ ID NO: 16;
   (ii) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;
   (iii) a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and
   (iv) a beta-xylosidase encoded by a polynucleotide that hybridizes under at least high stringency conditions, very high stringency conditions, with the mature polypeptide coding sequence of SEQ ID NO: 15 or the full-length complement thereof.
59. Use of the rinsing aid of paragraphs 52-58 for ADW.

### EXAMPLES

### Assays

### Wash Assay I—Full Scale Wash

The enzyme preparation was tested using a full scale wash in a Miele G4300 SCU automatic dishwashing machine. Washing program used was Universal 50° C., using tap water with water hardness 20° dH and with a total washing time of about 90 minutes. The washing programme comprises a rinsing cycle, a washing cycle followed by two rinsing cycles.

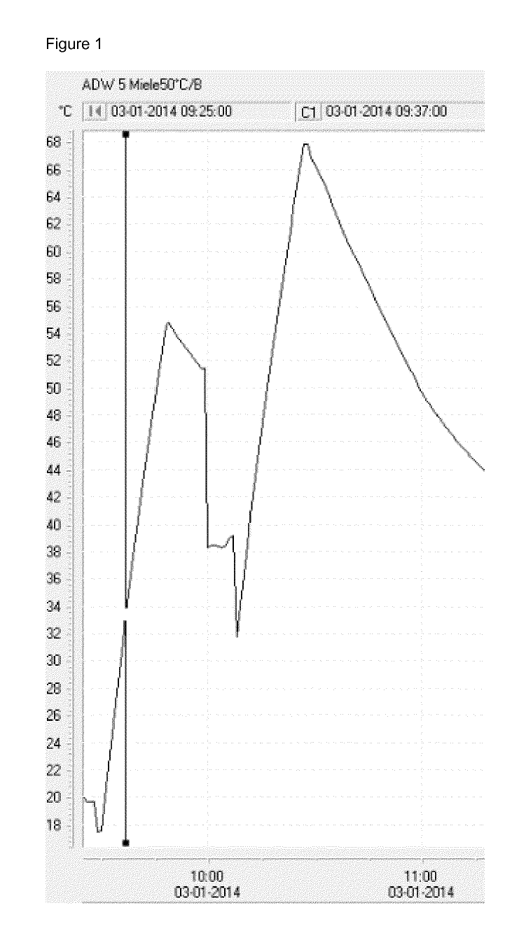

FIG. 1 shows the temperature in the automatic dish wash machine versus the washing time. The temperature profile was measured for the Universal 50° C. programme during one the washes performed during the experiment. From the figure it is seen that in the beginning of the wash program, cold water is coming in and decreasing the temperature somewhat and after that the heat up is starting and reaching up to about 50-54° C. The main wash at 50-54° C. continues for about 20-25 minutes after that the water is drained and the temperature decreases. Then clean tap water is supplied and a small temperature increase is seen. This corresponds to the first rinse cycle. After the first rinse cycle the rinse water is drained and clean tap water is supplied. The water is heated to about 68 C. The rinse water is drained after a few minutes, ending the second rinse cycle. The temperature in the drying phase slowly decreases. The wash cycle is finished after a total of 90 minutes.

For rinsing tap water with water hardness 20° dH was used. The amount of water in the main wash was 5.4 liter.

The washing was completed with either tap water or with the commercially available ADW liquid detergent (Persan) was used. In addition 50 grams of soil was added into the machine before start. The soil was prepared as shown in appendix 3 on page 44 of SÖFW-Journal, volume 132, No 8-2006.

The wash was completed without dishware in the dishwashing machine.

### Wash Assay II—One Hour Beaker Wash for 5 Days

On day 1 add a measured amount of cellulosic material to a beaker (1 L) and wash for one hour with a wash liquor comprising water and/or dishwashing composition at 40° C. in a beaker while mixing slowly. Simultaneously add a measured amount of cellulosic material to a beaker (1 L) and wash for one hour with a wash liquor comprising water and/or dishwashing composition and enzyme preparation and/or microorganism at 40° C. in a beaker while mixing slowly. Remove the wash liquor from both beakers by filtering but keep the cellulosic material in the beakers and put a lid on the beaker. Let it stand for 23 h at room temperature.

On day 2 wash again one hour with or without any enzyme preparation at 40° C. The cellulosic material that was washed a wash liquor comprising enzyme preparation on day 1 is washed with a similar wash liquor again. Similarly the cellulosic material that was washed without enzyme preparation on day 1 is washed with similar wash liquor again. Remove the wash liquor from the beaker by filtering but keep the cellulosic material in the beaker and put a lid on the beaker. Let it stand for 23 h at room temperature.

This process is repeated on day 3, 4 and 5 corresponding to five days in total. On day 5 the beaker is not left for standing 23 hours after washing, but the cellulosic material is removed from the beaker by filtering and left visually to be evaluated. A trained test person evaluates the amount of cellulosic material that is left after washing with enzymes in percentage of the amount of cellulosic material that is left after washing without enzymes. Further the cellulosic material is weighed.

### Wash Assay III—48 Hours Beaker Wash

Add a measured amount of cellulosic material to a beaker (1 L) and wash for 48 hours at 40° C. with a wash liquor comprising water and/or dishwashing composition in the beaker while mixing slowly. Simultaneously add a measured amount of cellulosic material to a beaker and wash for 48 hours at 40° C. with a wash liquor comprising water and/or dishwashing composition and enzyme preparation and/or microorganism while mixing slowly. Remove the wash liquor by filtrating the water so that cellulosic material left can removed and evaluated. The cellulosic material left is visually to be evaluated. A trained test person evaluates the amount of cellulosic material that is left after washing with enzymes in percentage of the amount of cellulosic material that is left after washing without enzymes. Further the cellulosic material is weighed.

### Wash Assay IV—Microorganism Wash in Beaker

Bushnell Haas Medium (+0.08% Yeast Extract):

Ingredients per 1000 ml

| | | |
|---|---|---|
| Magnesium Sulfate (MgSO4) | 0.2 | g |
| Calcium Chloride (CaCl2) | 0.02 | g |
| Monopotassium Phosphate (KH2PO4) | 1 | g |
| Diammonium Hydrogen Phosphate ((NH4)2HPO4) | 1 | g |
| Potassium Nitrate (KNO3) | 1 | g |
| Ferric Chloride (FeCl3) | 0.05 | g |
| Yeast extract | 0.8 | g |
| Distilled water | 1000 | ml |

The test conditions are the following:
1. Prepare 1 L of Bushnell Haas medium supplemented with 0.08% of yeast extract in beaker (1 L/beaker).
2. Place the cellulosic material in the beaker.
3. Prepare an inoculum of bacterial spores (dormant form) by diluting a liquid concentrate of microorganism 1000 times and inoculate 1 ml of this dilution in the beaker.
4. After inoculating the bacteria in the beaker it is left standing at 35° C. for 96 hours while mixing slowly.
5. The cellulosic material left is visually to be evaluated. A trained test person evaluates the amount of cellulosic material that is left after washing with the microorganism in percentage of the amount of cellulosic material that is left after washing without the microorganism. Further the cellulosic material is weighed.

### Composition of Various Detergent Compositions

| Detergent | Composi- | Composi- | Composi- |
|---|---|---|---|
| component | tion 1 g/L | tion 2 g/L | tion 3 g/L |
| MGDA | 1.68 | 0.77 | 1.65 |
| Sodium Carbonate | 0.67 | 0.67 | 0.66 |
| Sodium percarbonate | 0.38 | 0.38 | 0.33 |
| Sodium disilicate | 0.21 | 0.17 | 0.17 |
| Sodium sulphate | 1.11 | 1.11 | 1.06 |
| Acusol 588 (liq) | 0.45 | 0.45 | — |
| TAED/MnOx | 0.10 | 0.10 | 0.15 |
| Non ionic surfactant | 0.17 | 0.17 | 0.17 |
| Sokalan ® CP5 (liq) | — | — | 0.42 |

Composition of ADW liquid detergent (Persan): Aqua, Tetrasodium dicarboxymethyl glutamate, Sorbitol, Sodium citrate, sodium poliacrilate (salt solution in water), 2 propylheptanol ethoxylated polymer, Citric acid, Sodium hydroxide, Peg-10, Propylene glycol, Xanthan gum, Glycerin, Sodium diethylenetriamine pentamethylene phosphonate, Subtilisina, Zinc chloride, Parfum, Benzotriazole, Amilasa a-, Limoneno, Disubtituted alaninamide, Colorant Methylchloroisothiazolinone/methylisothiazolinone.

### Enzyme Preparation Capable of Degrading Cellulosic Material

The enzyme preparation capable of degrading cellulosic material comprises a blend of an Aspergillus fumigatus GH10 xylanase and Aspergillus fumigatus beta-xylosidase with a Trichoderma reesei cellulase preparation containing Aspergillus fumigatus cellobiohydrolase I, Aspergillus fumigatus cellobiohydrolase II, Aspergillus fumigatus beta-glucosidase variant, and Penicillium sp. (emersonii) GH61 polypeptide. The enzyme preparation can be produced as described in examples 1-19 of WO 2013/028928, which is hereby incorporated by reference.

### Pretreatment of Spinach

### Pretreatment Method I:

5 gram frozen whole leave spinach (“Værsgo” helbladet spinat, bought in Superbest, Copenhagen, Denmark) was pre-treated in a beaker with balsam vinegar (Chatel vinaigre balsamique de modène (Aceto balsimoco di modena I.G.P.) 6%) for 30 minutes at room temperature (20° C.) and distributed in the dishwashing machine.

The pH of the vinegar was pH3.

### Pretreatment Method II

A measured amount of frozen whole leave spinach (“Værsgo” helbladet spinat, bought in Superbest, Copenhagen, Denmark) was pre-treated in a beaker with citric acid at pH 4 for 10 min at room temperature in a beaker.

### Pretreatment Method III

A measured amount of frozen whole leave spinach (“Værsgo” helbladet spinat, bought in Superbest, Copenhagen, Denmark) was pre-treated in a beaker with HCl at pH 3 for 60 min at room temperature.

### Pretreatment Method IV

A measured amount of frozen whole leave spinach (“Værsgo” helbladet spinat, bought in Superbest, Copenhagen, Denmark) was pre-treated at 100° C. in a beaker with citric acid at pH 4 for 10 minutes.

### Pretreatment Method V

A measured amount of frozen whole leave spinach (“Værsgo” helbladet spinat, bought in Superbest, Copenhagen, Denmark) was pre-treated with water at 100° C. in a beaker for 10 minutes.

Evaluation of Cellulose Amount Left after Wash

The spinach was collected from the filter in the bottom of the dishwashing machine and it was determined how much spinach that was left after the wash.

### Example 1

Wash assay I was used to test the effect of using the enzyme preparation in ADW at various degrees of water hardness.

As detergent the liquid ADW detergent from Persan in dosage 32 gram/wash was used. Further, 1.28 gram of protease (Blaze®, supplied by Novozymes A/S, SEQ ID NO: 25) and 0.256 gram amylase (Stainzyme Plus®, supplied by Novozymes A/S, SEQ ID NO: 26) were added per wash.

A first wash was then completed as described in wash assay I.

The spinach was collected from the filter in the bottom of the dishwashing machine and it was determined visually how much spinach that was left after the wash. The spinach was then left in the washing machine.

On days 2-5, one wash/day was completed. The remains of spinach left after wash the day before were still in the dishwashing machine. After each wash the spinach were collected from the filter in the bottom of the dishwashing machine and it was determined visually how much spinach that was left after the wash.

For comparison, a similar wash schedule was completed exactly as described above and with 1.6 gram added of the enzyme preparation (supplied by Novozymes A/S) capable of degrading cellulosic material.

Results:

| | % spinach left | % spinach left |
|---|---|---|
| | Without enzyme | With enzyme |
| Wash number | preparation | preparation |
| Day 1 - after | 100 | 100 |
| pretreatment | | |
| Day 1 | 100 | 100 |
| After washing | | |
| Day 2 | 100 | 70 |
| Day 3 | 80 | 50 |
| Day 4 | 70 | 10 |
| Day 5 | 60 | 10 |

### Example 2

No detergent composition was used. Only tap water and enzymes were used. The pH of the water was adjusted to pH 7-8 in the machine by titrating with 4M HCL and/or 4M NaOH. Further 0.8 gram Blaze® Evity 100T (supplied by Novozymes A/S, SEQ ID NO: 25) and 0.16 gram Stainzyme Plus® Evity 12T (supplied by Novozymes A/S, SEQ ID NO: 26) was added. A first wash was then completed as described in wash assay I.

The spinach was collected from the filter in the bottom of the dishwashing machine and it was determined visually how much spinach that was left after the wash. The spinach was then left in the washing machine.

On days 2-5, one wash/day was completed. The remains of spinach left after wash the day before were still in the dishwashing machine. After each wash the spinach were collected from the filter in the bottom of the dishwashing machine and it was determined visually how much spinach that was left after the wash.

For comparison, a similar wash schedule was completed exactly as described above and with 1.6 gram added of the enzyme preparation (supplied by Novozymes A/S) capable of degrading cellulosic material.

Results:

| | % spinach left | % spinach left |
|---|---|---|
| | Without enzyme | With enzyme |
| Wash number | preparation | preparation |
| Day 1 - after | 100 | 100 |
| pretreatment | | |
| Day 1 | 100 | 100 |
| After washing | | |
| Day 2 | 100 | 100 |
| Day 3 | 90 | 80 |
| Day 4 | 80 | 10 |
| Day 5 | 80 | 0 |

### Example 3

5 gram/L of spinach was washed according to wash assay II and the amount of spinach left was evaluated by a trained test person.

### Example 4

5 gram/L of spinach or avocado was washed according to wash assay III with and without pretreatment of the spinach. The amount of spinach left was evaluated by a trained test person.

| | Visual scoring - % spinach | Visual scoring - % avocado |
|---|---|---|
| | left versus wash without | left versus wash without |
| | enzyme preparation | enzyme preparation |
| no pretreatment | 60 | 50 |
| 10 min/pH 4 | 40 | not evaluated |
| pretreatment | | |
| 60 min/pH 3 | 30 | not evaluated |
| pretreatment | | |

### Example 5

Bacillus subtilis SB3175 was prepared according to wash assay IV and added to and washed with 5 gram of spinach as described in wash assay IV. The result is evaluated as described and data is shown below.

| | Visual scoring - % spinach left versus wash |
|---|---|
| | without microorganism preparation |
| Bacillus subtilis SB3175 | 50% |

1-15. (canceled)

16. A dishwashing composition comprising a builder and one or more enzymes capable of degrading cellulosic material, wherein the one or more enzymes capable of degrading cellulosic material is an enzyme preparation comprising:
a) a cellobiohydrolase I;
b) a cellobiohydrolase II;
c) a beta-glucosidase or variant thereof; and
d) a GH61 polypeptide having cellulolytic enhancing activity; or homologs thereof.

17. The dishwashing composition of claim 1, wherein the one or more enzymes capable of degrading cellulosic material is an enzyme preparation comprising:
a) an Aspergillus fumigatus cellobiohydrolase I;
b) an Aspergillus fumigatus cellobiohydrolase II;
c) an Aspergillus fumigatus beta-glucosidase or variant thereof; and
d) a Penicillium sp. GH61 polypeptide having cellulolytic enhancing activity; or homologs thereof.

18. The dishwashing composition of claim 1, wherein
(a) the Aspergillus fumigatus cellobiohydrolase I or homolog thereof of the enzyme preparation is selected from the group consisting of:
(i) a cellobiohydrolase I comprising or consisting of the mature polypeptide of SEQ ID NO: 2;
(ii) a cellobiohydrolase I comprising or consisting of an amino acid sequence having at least 70% sequence identity to the mature polypeptide of SEQ ID NO: 2;
(iii) a cellobiohydrolase I encoded by a polynucleotide comprising or consisting of a nucleotide sequence having at least 70% sequence identity to the mature polypeptide coding sequence of SEQ ID NO: 1; and
(iv) a cellobiohydrolase I encoded by a polynucleotide that hybridizes under high stringency conditions with the mature polypeptide coding sequence of SEQ ID NO: 1 or the full-length complement thereof;
(b) wherein the Aspergillus fumigatus cellobiohydrolase II or homolog thereof is selected from the group consisting of:
(i) a cellobiohydrolase II comprising or consisting of the mature polypeptide of SEQ ID NO: 4;
(ii) a cellobiohydrolase II comprising or consisting of an amino acid sequence having at least 70% sequence identity to the mature polypeptide of SEQ ID NO: 4;
(iii) a cellobiohydrolase II encoded by a polynucleotide comprising or consisting of a nucleotide sequence having at least 70% sequence identity to the mature polypeptide coding sequence of SEQ ID NO: 3; and
(iv) a cellobiohydrolase II encoded by a polynucleotide that hybridizes under high stringency conditions with the mature polypeptide coding sequence of SEQ ID NO: 3 or the full-length complement thereof;
(c) wherein the Aspergillus fumigatus beta-glucosidase or homolog thereof is selected from the group consisting of:
(i) a beta-glucosidase comprising or consisting of the mature polypeptide of SEQ ID NO: 6;
(ii) a beta-glucosidase comprising or consisting of an amino acid sequence having at least 70% sequence identity to the mature polypeptide of SEQ ID NO: 6;
(iii) a beta-glucosidase encoded by a polynucleotide comprising or consisting of a nucleotide sequence having at least 70% sequence identity to the mature polypeptide coding sequence of SEQ ID NO: 5;
(iv) a beta-glucosidase encoded by a polynucleotide that hybridizes under high stringency conditions with the mature polypeptide coding sequence of SEQ ID NO: 5 or the full-length complement thereof; and
(v) a beta-glucosidase variant comprising a substitution at one or more positions corresponding to positions 100, 283, 456, and 512 of the mature polypeptide of SEQ ID NO: 6, wherein the variant has beta-glucosidase activity; and
(d) wherein the Penicillium sp. GH61 polypeptide having cellulolytic enhancing activity or homolog thereof is selected from the group consisting of:
(i) a GH61 polypeptide having cellulolytic enhancing activity comprising or consisting of the mature polypeptide of SEQ ID NO: 8;
(ii) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
(iii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
(iv) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.

19. The dishwashing composition of claim 1, wherein the beta-glucosidase variant of the enzyme preparation comprises one or more (several) substitutions selected from the group consisting of G142S, Q183R, H266Q, and D703G.

20. The dishwashing composition of claim 1, wherein the enzyme preparation further comprises one or more enzymes selected from the group consisting of:
a) an Aspergillus fumigatus xylanase or homolog thereof,
b) an Aspergillus fumigatus beta-xylosidase or homolog thereof; or
c) a combination of (i) and (ii);
wherein the Aspergillus fumigatus xylanase or homolog thereof is selected from the group consisting of:
i) an Aspergillus fumigatus xylanase comprising or consisting of the mature polypeptide of SEQ ID NO: 10, SEQ ID NO: 12, or SEQ ID NO: 14;
ii) a xylanase comprising or consisting of an amino acid sequence having at least 70% sequence identity to the mature polypeptide of SEQ ID NO: 10, SEQ ID NO: 12, or SEQ ID NO: 14;
iii) a xylanase encoded by a polynucleotide comprising or consisting of a nucleotide sequence having at least 70% sequence identity to the mature polypeptide coding sequence of SEQ ID NO: 9, SEQ ID NO: 11, or SEQ ID NO: 13; and
iv) a xylanase encoded by a polynucleotide that hybridizes under high stringency conditions with the mature polypeptide coding sequence of SEQ ID NO: 9, SEQ ID NO: 11, or SEQ ID NO: 13; or the full-length complement thereof; and
wherein the Aspergillus fumigatus beta-xylosidase or homolog thereof is selected from the group consisting of:
(i) a beta-xylosidase comprising or consisting of the mature polypeptide of SEQ ID NO: 16;
(ii) a beta-xylosidase comprising or consisting of an amino acid sequence having at least 70% sequence identity to the mature polypeptide of SEQ ID NO: 16;
(iii) a beta-xylosidase encoded by a polynucleotide comprising or consisting of a nucleotide sequence having at least 70% sequence identity to the mature polypeptide coding sequence of SEQ ID NO: 15; and
(iv) a beta-xylosidase encoded by a polynucleotide that hybridizes under high stringency conditions with the mature polypeptide coding sequence of SEQ ID NO: 15 or the full-length complement thereof.

21. The dishwashing composition of claim 1, wherein the dishwashing composition comprises at least one enzyme in addition to the enzymes in the enzyme preparation.

22. The dishwashing composition of claim 1, wherein the composition is for used in ADW.

23. The dishwashing composition of claim 1, wherein the composition further comprises a microorganism capable of degrading cellulosic material.

24. The dishwashing composition of claim 23, wherein the microorganism is Bacillus subtilis SB3175.

25. A washing method comprising exposing the dish ware to wash liquor comprising the dishwashing composition of claim 16.

26. The method of claim 25, wherein the method comprises the steps of:
a) Exposing the dishes to an aqueous solution having a pH below 4;
b) Exposing the dishes to a wash liquor comprising the dishwashing composition; and
c) Rinsing the dishes with water or an aqueous solution comprising a rinsing aid;
wherein step a) is carried out before step b) or step a) is carried out simultaneously with step b) or step c).

27. The method of claim 25, wherein the washing method is hand dishwashing or ADW.

28. A cleaning method for cleaning the interior of an automated dish washing machine, which method comprises exposing the interior of the dish washing machine to the dishwashing composition of claim 16.

29. A rinsing aid for use in an automatic dish washing method, wherein the rinsing aid is capable of lowering the pH below 4 during at least a period of a rinse cycle in an automated dishwashing process.
